(19) United States
(12) Patent Application Publication
(10) Pub. No.: US 2023/0023475 A1
(43) Pub. Date: Jan. 26, 2023

(54) SYSTEMS AND METHODS FOR STIMULATION

(71) Applicant: Lungpacer Medical Inc., Vancouver (CA)

(72) Inventors: Thiago G. BASSI, Burnaby (CA); Douglas G. EVANS, Downingtown, PA (US)

(73) Assignee: Lungpacer Medical Inc., Vancouver, BC (CA)

(21) Appl. No.: 17/758,362

(22) PCT Filed: Jan. 13, 2021
(86) PCT No.: PCT/IB2021/050214

§ 371 Date: Jul. 5, 2022

Related U.S. Application Data
(60) Provisional application No. 62/960,299, filed on Jan. 13, 2020.

(51) Int. Cl. A61N 1/36; A61B 5/08; A61B 5/00; A61M 16/06; A61M 16/04; A61N 1/05

(52) U.S. Cl. CPC A61N 1/3601; A61B 5/0816; A61B 5/4836; A61M 16/0666; A61M 16/0459; A61N 1/0546; A61M 2205/054

(57) ABSTRACT

A stimulation device may include a tube, where the tube defines a lumen therein and a gas outlet. The gas outlet may be configured to allow gas to pass from the lumen to an exterior of the tube. The stimulation device may also include one or more first electrodes associated with the tube, one or more second electrodes, and a nasal cavity interface proximal of the gas outlet and the one or more first electrodes.

### CROSS-REFERENCE TO RELATED APPLICATION

This application claims priority to U.S. Provisional Application No. 62/960,299, filed on Jan. 13, 2020, which is hereby incorporated by reference in its entirety. All publications and patent applications mentioned in this specification are herein incorporated by reference in their entirety to the same extent as if each individual publication or patent application was specifically indicated to be incorporated by reference. For example, embodiments of the present disclosure may be used in combination with one or more systems, catheters, apparatuses, and electrodes described in U.S. Pat. Nos. 9,242,088, 9,333,363, 9,776,005, 10,039,920, U.S. Pat. Pub. 2019/0038894, and/or U.S. Pat. Pub. 2020/0147364; the disclosures of which are hereby incorporated by reference.

### TECHNICAL FIELD

The embodiments of this disclosure generally relate to methods and devices (including systems) for the stimulation of nerves, muscles, and/or other body tissue. More specifically, embodiments of the present disclosure include methods and systems including a nasal stimulator.

### BACKGROUND

Patients in hospital Intensive Care Units (ICUs) may experience impairment in their ability to breathe volitionally due to their underlying disease condition. Such patients may require positive pressure mechanical ventilation (MV) and/or other means of External Respiratory Support, collectively ERS, to provide ventilatory assistance. ERS is often used in combination with sedation in the ICU to provide artificial ventilation for these critically ill individuals. Additionally, many patients undergoing surgery under general anesthesia, for example in hospital Operating Rooms (ORs), or procedures requiring anesthesia or sedation, for example in hospital Emergency Rooms (ERs), commonly require ERS for ventilatory assistance while anesthetized or sedated.

Although mechanical ventilation is a life-sustaining modality, when combined with sedation or anesthesia, it interferes with active contraction of the diaphragm. Prolonged totally controlled mechanical ventilation can result in the complete absence of neural activation and mechanical activity of the diaphragm and has been shown to induce muscle atrophy, proteolysis, and reactive oxygen species liberation, leading to rapid losses in diaphragmatic function, a syndrome known as Ventilator-Induced Diaphragmatic Dysfunction (VIDD). These patients are also known to experience higher levels of lung, brain, heart and other organ injury. They are also at higher risk of other comorbidities including infection and sepsis, and each day on ERS increases the risk of death.

Approximately 15 million ICU patients require mechanical ventilation annually. Further, approximately a third of those patients will require prolonged weaning to overcome ventilator dependency. Most patients who require diaphragm weaning exhibit diaphragmatic atrophy and dysfunction. Overall, patients requiring mechanical ventilation end up with prolonged and lengthy ICU/hospital stays, higher healthcare costs, poor long-term functional outcomes, and increased respiratory complications and mortality.

During a natural breathing cycle, physiological connections facilitate communication between components of a subject's respiratory system. During traditional respiration therapies, ERS, or other means of respiratory assistance, atypical physiological reactions may result, as compared to innate, un-aided respiration. The atypical physiological reactions may have deleterious consequences for the subject and/or the efficacy of the respiration assistance.

### SUMMARY

Embodiments of the present disclosure relate to, among other things, systems, devices, and methods for providing respiratory support. Embodiments include systems and devices for applying stimulation to one or more anatomical targets. Embodiments of the systems and methods described herein, may be used with alternatives and/or supplements to MV and/or may incorporate ERS, such as, for example, stimulation of respiratory nerves and/or respiratory muscles. Each of the embodiments disclosed herein may include one or more of the features described in connection with any of the other disclosed embodiments.

In one example, a stimulation device comprises a tube defining a lumen therein and a gas outlet, one or more first electrodes associated with the tube, one or more second electrodes; and a nasal cavity interface proximal of the gas outlet and the one or more first electrodes. The gas outlet may be configured to allow gas to pass from the lumen to an exterior of the tube.

Any of the systems or methods disclosed herein may include any of the following features. A gas hose may be connected to the nasal cavity interface. The gas hose may include a distal end that allows gas to flow form the gas hose to the gas outlet, and a proximal end configured to receive gas into the gas hose from a gas source. One or more metal leads may be connected to the one or more first electrodes, wherein the one or more metal leads are disposed within the nasal cavity interface and within the gas hose. An inlet port may be configured to allow gas to pass from the exterior of the stimulation device to the interior of the stimulation device. The gas outlet may be a first gas outlet, and the stimulation device may also include a second gas outlet configured to allow gas to pass from the lumen to the exterior of the stimulation device. The stimulation device may also include a first occlusion device, a second occlusion device, or both. Either or both occlusion devices, may be configured to expand upon receipt of a fluid transmitted through the nasal cavity interface. The first occlusion device may be between the first gas outlet and the second gas outlet. The first occlusion device may support the one or more first electrodes, and the second occlusion device may support the one or more second electrodes. The tube of the stimulation device may be a first nasal tube and the stimulation device may also include a second nasal tube. The one or more first electrodes may be disposed on a surface of the first nasal tube. The one or more second electrodes may be disposed on a surface of the second nasal tube. The first gas outlet may be disposed on a distal end of the first nasal tube. The second gas outlet may be disposed on a distal end of the second nasal tube. The one or more first electrodes may include a first line of electrodes perpendicular to the first gas outlet, and a second line of electrodes parallel to the first line of electrodes. The one or more second electrodes may include a third line of electrodes perpendicular to the second gas outlet, and a fourth line of electrodes parallel to the third line of electrodes. The stimulation device may further include a dermal patching supporting the one or more second electrodes and configured for placement on skin. The stimulation device may also include a sensor and a controller in communication with the sensor, the one or more first electrodes, and the one or more second electrodes. The sensor may be supported on an endoscope. The controller may be configured to adjust an amount of gas passing through the gas outlet, analyze data collected by the sensor to determine a breath cycle, adjust an amount of gas passing through the gas outlet based on the determined breath cycle, deliver signals to the one or more first electrodes and the one or more second electrodes such that an electrical field is generated between the one or more first electrodes and the one or more second electrodes, or a combination thereof.

### BRIEF DESCRIPTION OF THE DRAWINGS

The accompanying drawings, which are incorporated in and constitute a part of this specification, illustrate non-limiting embodiments of the present disclosure and together with the description serve to explain the principles of the disclosure.

FIG. 1A illustrates a nasal stimulator affixed to a subject, according to one or more embodiments;

FIG. 1B illustrates a nasal stimulator affixed to a subject, according to one or more embodiments;

FIG. 2 illustrates a nasal stimulator affixed to a subject, according to one or more embodiments;

FIG. 3A illustrates a nasal stimulator, according to one or more embodiments; and

FIG. 3B illustrates the nasal stimulator of FIG. 3A, rotated 90° about a longitudinal axis.

### DETAILED DESCRIPTION

Further aspects of the disclosures and features of example embodiments are illustrated in the appended drawings and/or described in the text of this specification and/or described in the accompanying claims. It may be understood that both the foregoing general description and the following detailed description are exemplary and explanatory only and are not restrictive of the invention, as claimed.

As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

Additionally, the term “exemplary” is used herein in the sense of “example,” rather than “ideal.” As used herein, the term “proximal” means a direction closer to an operator and the term “distal” means a direction further from an operator. The term “approximately” or like terms (e.g., “about,” “substantially”) encompass values within 10% of the stated value.

Embodiments of the present disclosure may induce nasal nerve fiber activity during artificial ventilation or non-nasal respiration. One or more embodiments include a nasal stimulation therapy (NST) system that may provide beneficial physiologic responses related to compliance of the respiratory system (e.g. lungs, etc.), improvements in cognition and memory, stabilization of mood, and modulation of neurogenesis, among others. An NST system may be used alone or in combination with external respiratory support (e.g., mechanical ventilation, respiratory muscle stimulation, etc.). In some embodiments, the NST system does not provide the primary respiratory support of the patient, but is coordinated with the respiratory cycle and one or more other systems providing primary respiratory support. Methods and systems of this disclosure may modulate hippocampus neurogenesis by, for example, physiologically stimulating the olfactory bulb in coordination with invasive or non-invasive ventilation (e.g., mechanical ventilation or respiratory muscle stimulation). Such modulation of hippocampus neurogenesis may result in better cognitive outcomes in critical and non-critical patients.

Physiological connections exist between the nose and the lungs of a subject (e.g., a patient). The classic nasobronchial reflex, a component of the diving reflex, may lead to suppression of respiration (apnea), laryngospasm, and/or bronchoconstriction. The nasocardiac component of the diving reflex may include bradycardia, decreased cardiac output, and/or vasoconstriction in the skin, muscles, and gastrointestinal and renal circulation systems. The reflex may effectively keep water out of the lungs and/or decrease oxygen consumption while maintaining cerebral perfusion.

Nasal inhalation may affect the pulmonary system. For example, nasal inhalation of dust, smoke, ammonia, phenylethylacetate, perfume, sulfur dioxide, and other water-soluble chemicals can induce immediate bronchoconstriction with cessation of respiration in the expiratory phase due to relaxation of inspiratory muscles. Further, nasal stimulation with cold dry air may decrease proximal tracheal mucosal blood flow, without affecting airflow.

Activation of nasal afferent receptors may lead to significant bronchial obstruction and cardio depressor reflexes. Mechanosensitive nasal receptors can stimulate nasobronchial reflexes. Afferent and efferent pathways that may be related to these physiological reflexes may include the maxillary branch of the trigeminal nerve, the mandibular branch of the trigeminal nerve, the vagus nerve, and/or other connected afferents. Targets for stimulation may include any of the connected afferents, olfactory bulb receptors, the prefrontal cortex, suprachiasmatic nucleus, the hypothalamus, respiratory motor cortex and/or the brain stem.

Afferent nasal, tracheobronchial, and inspiratory muscle mechanoreceptors may participate in the coordination of inspiratory efforts. Dynamic inspiratory load, a measure of the changing muscular effort during inhalation, is active in awake subjects, and is modified by, for example, posture, origin of breath (mouth or nose breathing), and exercise.

In some instances, nose breathing may increase hippocampus activity, such as, for example, hippocampus activity in synchrony with inspiration. Further, nose breathing may affect mood and stress reactions. In contrast, mouth breathing may reduce hippocampal activity and/or increase cellular hippocampal stress. Systems and methods of the present disclosure may involve modulating nose airflow, for example, to improve the hippocampus environment and connectivity.

A subject's innate breathing cycle may work in synchrony with the central nervous system activity, for example, during nose breathing. During non-nasal respiration (e.g., mouth breathing) or external respiratory support (e.g., mechanical ventilation), synchronization with central nervous system activity may be lost. Lack of synchronization may lead to deleterious effects to tissue (e.g., neural tissue of the central nervous system).

According to one or more embodiments, nasal nerve fibers may be stimulated via, for example, airflow, electrical stimulation, optical activation, magnetic stimulation, pressure change (e.g., a change in nasal cavity pressure), or a combination thereof. In some embodiments, nasal nerve fibers may be stimulated in synchrony with the breathing cycle of a subject, such as for example, an innate breathing cycle or a breathing cycle regulated by one or more external respiratory support systems. Stimulating nasal nerve fibers in coordination with the breathing cycle of a subject may provide improvements in cognition, memory, mood stabilization, and/or the modulation of neurogenesis. Further, stimulating nasal nerve fibers in coordination with the breathing cycle may provide beneficial physiologic responses related to compliance of the respiratory system (e.g. lungs, airways), such as, for example, increasing the compliance of the lungs and/or airways. Increasing compliance of the respiratory system may reduce the overall work of breathing which may provide further physiological benefits to the subject. Further, some forms of respiratory support, for example positive pressure mechanical ventilation, may cause lung injury. For example, as positive pressure is used to expand and fill both airways and lungs, increasing the compliance of the respiratory system can potentially make it easier to ventilate a patient and thereby reduce the potential for injury.

Some embodiments, systems, and methods described herein may utilize cyclical stimulus that is synchronized with the respiratory cycle. For example, NST may be delivered for each breath cycle, for a series of breath cycles (e.g., random breath cycles, periodic breath cycles, or pre-determined breath cycles) over a period of time. The stimulation may be consistent between breath cycles where stimulation is delivered or may be varied (e.g., patterned in increasing or decreasing magnitude based on a patient's need). For example, nasal stimulation can be cyclically coordinated with each breath to trigger increased lung compliance and thereby create a physiological state which is more compatible with external respiratory support (e.g. mechanical ventilation, high flow O₂, CPAP, Bi-Pap, etc.) or other respiratory support technologies such as, for example, nerve stimulation (e.g. phrenic nerve stimulation), respiratory muscle (e.g. diaphragm, intercostal, abdominal, etc.) activation systems, and/or extracorporeal membrane oxygenation. In addition or alternatively, the NST may be delivered noncyclically.

According to one or more embodiments, an NST system may include a controller, an energy source (e.g., an electrical source), a gas supply source, one or more sensors, and/or a nasal stimulator. The components of the NST system may communicate with each other via wired or non-wired connections (e.g. Wi-Fi, RF, Bluetooth, USB, etc.). The nasal stimulator may connect to one or more stimulation sources (e.g., a gas supply source and/or an electrical source).

Electrical sources may include alternating current (e.g., wall power), direct current (e.g., battery power), or other sources of electrical energy. The nasal stimulator may be configured to deliver electrical energy (discussed further below) at low frequencies (e.g., approximately 1 Hertz (Hz) to approximately 45 Hz) and/or high frequencies (e.g., approximately 10,000 Hz). The electrical energy may be delivered in pulses, where each pulse independently has a width of approximately 10 milliseconds (ms) to approximately 500 ms, and the total duration of each delivery of electrical stimulation is approximately 0.1 seconds to approximately 3.0 seconds (e.g., approximately 0.1 seconds to 2.0 seconds during inspiration and/or approximately 0.1 seconds to 3.0 seconds during expiration). The amplitude of delivered electrical energy may vary from approximately 1 milliampere (mA) to approximately 20 mA.

In one or more embodiments, the nasal stimulator may be connected to one or more gas supply sources, such as, for example, a source of room air, medical air, oxygen, and/or a gas mixture including oxygen and one or more other gases (e.g., nitrogen, argon, carbon dioxide, helium, etc.). Other gases, such as, for example, medical gases (e.g., nitrous oxide), pharmaceuticals (e.g. albuterol, etc.), and/or anesthesia can also be introduced into the gas supply. The nasal stimulator may be connected to a gas-cylinder, a compressed gas-line, and/or an ambient source. In some embodiments, the nasal stimulator may draw air from the surrounding environment (or other sources), and process, clean, filter, humidify, heat, and/or cool the air. The nasal stimulator may adjust the pressure and flow rate of the gas source as required for therapeutic use.

For example, the nasal stimulator may be configured to nasally deliver gas at a flow rate of approximately 5 liters per minute (L/min) to approximately 70 L/min, depending on the needs of the patient, such as, for example, approximately 30 L/min to approximately 50 L/min. The flow rates of gas delivered by the nasal stimulator may be constant or may be varied. For example, the flow rate may be modulated in synchrony with a respiratory cycle of a patient, such as, for example, a respiratory cycle that includes an inspiration phase which has a duration of approximately 1.0 second to approximately 3.0 seconds, and an expiratory phase which has a duration of approximately 3.0 seconds to approximately 5.0 seconds. Delivery of gas via the nasal stimulator may be at a higher flow rate during an inspiration phase as compared to the flow rate of gas delivered during an expiration phase. Gas may be delivered via the nasal stimulator such that transnasal pressures are approximately 40 pascal (Pa) to approximately 80 Pa, or even less than approximately 40 Pa.

The one or more stimulation sources may communicate with a controller that can control the supply of nasal stimulation energy (e.g., gas flow and/or electrical current) to the nasal stimulator. Further, the controller may communicate with one or more sensors. Data collected from the one or more sensors may be used to adjust one or more parameters of stimulation. This adjustment may be performed by the controller, another unit or system, or a user. In some embodiments, data may be received/exchanged with another device (e.g., a diagnostic device, a therapeutic device, etc.). The other device may be in communication with the patient. In some embodiments, data may be exchanged with an external respiratory support system and/or one or more sensors connected to an external respiratory support system.

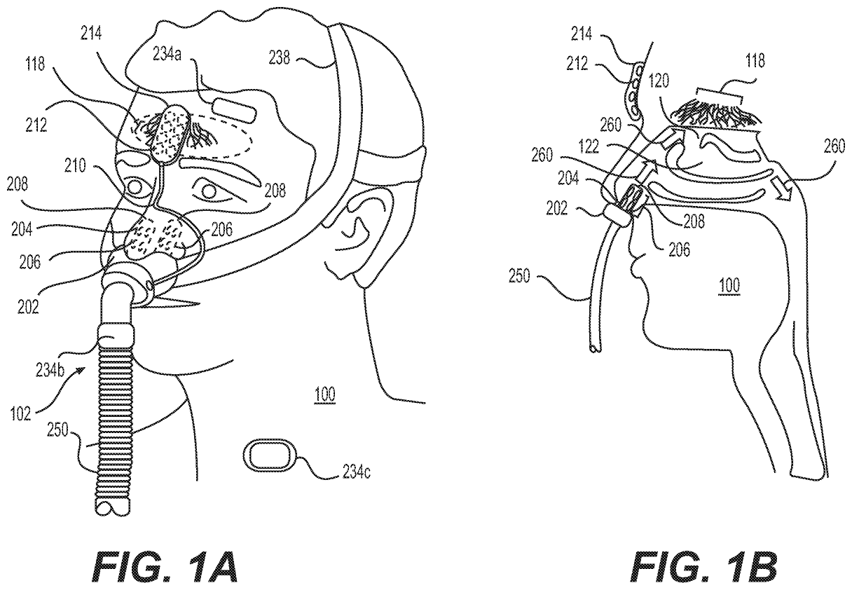

FIG. 1A shows a nasal stimulator 102 affixed to a subject 100, according to one or more embodiments. For example, nasal stimulator 102 may include an attachment means 238 that may secure or affix nasal stimulator 102 to subject 100. For example, attachment means 238 may include one or more straps affixed to another component of nasal stimulator 102, such as, for example, a nasal cavity interface 202, and the head of subject 100. The nasal cavity interface 202 (including, for example, a nasal mask or cannula) may be configured to be inserted into the nostrils of subject 100 or otherwise form a seal with the nasal passages of subject 100. The nasal cavity interface 202 may form an air-tight seal with the nasal passages of the subject 100. In some embodiments, the nasal cavity interface 202 may form a seal with the nasal passages of the subject 100 that is not air-tight (e.g., may allow gas to escape the nasal passages). For example, nasal cavity interface 202 may limit pressure levels by allowing gas to escape the nasal passages.

Additionally, nasal cavity interface 202 may form an interface between nasal tubes 206 and a gas hose 250. For example, gas may pass from a gas source (not pictured), through gas hose 250, to nasal cavity interface 202, through one or more nasal tubes 206, and out one or more outlets 208. The nasal tubes 206 and outlets 208 may be configured to deliver gas to one or more target afferents (e.g., olfactory bulb receptors 118), as described below. For example, outlets 208 may be configured to allow gas to pass from an interior of nasal stimulator 102 (e.g., a lumen of nasal stimulator 102) to an exterior of nasal stimulator 102. Further, one or more nasal electrodes 204 may be disposed on each of one or more nasal tubes 206. Electrical leads connecting nasal electrodes 204 to an energy source and/or a controller may pass through nasal cavity interface 202 and/or gas hose 250 to the energy source and/or controller (not pictured).

Nasal electrodes 204 may be disposed on a surface of nasal tubes 206 perpendicular to gas outlet(s) 208. Nasal electrodes 204 may be located at different radial positions of nasal tubes 206. Two or more nasal electrodes 204 of each nasal tube 206 may be arranged in rows/lines. For example, nasal electrodes 204 may be arranged in lines (e.g., parallel lines) at different radial positions about an axis of nasal tube 206). For example, a nasal tube 206 may include at least two nasal electrodes 204 aligned along a longitudinal axis of the nasal tube 206. Lines of longitudinally aligned nasal electrodes 204 may be spaced at different radial positions of nasal tube 206 (e.g., two lines spaced 180° apart, three lines spaced 120° apart, or four lines spaced 90° apart). In addition or alternatively, lines of longitudinally aligned nasal electrodes 204 may be spaced irregularly at different radial positions of nasal tube 206. In some embodiments, nasal electrodes 204 may be positioned lateral the medial line to direct energy to one or more non-medial targets.

According to one or more embodiments, nasal stimulator 102 may include a dermal patch 214. The dermal patch 214 may be affixed to the skin of a subject 100 via an adhesive or other means. Dermal patch 214 may include one or more dermal electrodes 212. Although FIG. 1A shows a dermal patch 214 including eight dermal electrodes 212, this is one example. Any number of electrodes may be included in dermal patch 214. The dermal electrodes 212 may be arranged in an array on or within dermal patch 214, for example, dermal electrodes 212 may be arranged in a series of rows, a grid, and/or another shape that allows for placement of one or more dermal electrodes 212 proximate to olfactory bulb receptors 118. According to one or more embodiments, such as, for example, the embodiment shown in FIG. 1A, dermal patch 214 may be ovular in shape. However, this is one example, and dermal patch 214 may be square, rectangular, elliptical, circular, trigonal, or other suitable shape that allows for dermal electrodes 212 to be arranged in a configuration proximate to one or more stimulation targets (e.g., olfactory bulb receptors 118). Dermal patch 214 may be flexible and able to conform to contours of subject 100. In some embodiments, dermal patch 214 may be resilient and resistant to deformation.

Dermal electrodes 212 may be configured to deliver energy, such as, for example, electromagnetic energy (e.g., infrared) or ultrasound. The dermal patch 214, and by extension, the dermal electrodes 212, may be connected to an energy source via, for example, a dermal patch lead 210. The dermal patch lead 210 may provide an electrical connection from a stimulation source to the dermal patch 214, via, for example, one or more leads disposed in gas hose 250 and/or nasal cavity interface 202. Dermal patch lead 210 may extend from dermal patch 214 to one or more other components of an NST system, such as, for example, nasal cavity interface 202 and/or an electrical energy source. Dermal patch lead 210 may extend on the exterior of subject 100, such as, for example, from a forehead of subject 100 to the nasal cavity of subject 100. In some embodiments, current may be delivered between nasal electrodes 204 and dermal electrodes 212. Such current may be used to target olfactory bulb receptors 118, one or more afferents of the nasal-bronchial reflex, the pre-frontal cortex, or the brain stem.

In some embodiments, an NST system may include one or more sensors, such as, for example, sensors 234a, 234b, and/or 234c, shown in FIG. 1A. The one or more sensors 234 may include a cognitive status sensor and/or a respiration sensor. For example, the NST system shown in FIG. 1A includes a cognitive status sensor 234a and two respiration sensors 234b and 234c. A cognitive status sensor (e.g., sensor 234a) may be used to measure the cognitive state of the patient and how the cognitive state may change over time.

A cognitive status sensor 234a may include a device configured to measure brain electrical activity. For example, cognitive status sensor 234a may include one or more electrodes configured to record the electrical activity of the patient's brain, over a period of time. Cognitive status sensor 234a, or a system in communication with sensor 234a, may generate an electroencephalogram (EEG) based on the recorded electrical activity of the brain. The generated EEGs may be used to characterize the “state” of the cognitive activity. For example, a ratio between alpha waves (e.g., approximately 8 Hz to approximately 12 Hz) and beta waves (e.g., approximately 12 Hz to approximately 40 Hz) may be determined. Without being limited by theory, alpha waves may be more prevalent in an EEG during a wakeful state and indicate a patient is quietly resting, while beta waves are more prevalent in an EEG when a patient is alert, attentive, and actively thinking. It is believed that an increase in alpha waves, for select periods of time, are beneficial to the health of patients. Measurements and ratios of other waves, such as gamma waves (e.g., approximately 40 Hz to approximately 100 Hz) may be used to provide insight into cognitive activity.

One or more respiration sensors (e.g., sensors 234b and/or 234c) may be used to measure the patient respiration. For example, a respiration sensor may be integrated into the gas flow line (e.g., sensor 234b between gas hose 250 and nasal cavity interface 202) or affixed to the skin of the subject 100 (e.g., sensor 234c). A respiratory sensor 234b may measure airflow, pressure, volume (e.g., tidal volume), or one or more other properties of respiration. One or more respiratory sensors 234c may include an accelerometer configured to determine timing of one or more components of a breath cycle (e.g., inspiration duration, inspiration pause, expiration duration, and/or expiration pause). Other sensors may assess various physiological conditions of the patient such as oxygenation levels (e.g. via pulse oximetry, blood gas analyzers, etc.), temperature, heart rate, blood pressure, insulin levels, etc.

Other sensors known in the art may be useful to provide input for managing, optimizing, and/or delivering therapy, or integrating with other devices. The sensors can be electrically coupled to energy sources and/or configured to include a battery. The sensors and other components of the system can communicate with each other via wired or non-wired connections (e.g. Wi-Fi, RF, etc.). In addition or alternatively, dermal patch 214 may include one or more sensors (e.g. electrodes, microphones, motion sensors, magnetic coils, thermistors, etc.) to detect signals from the subject 100. In some embodiments, a controller may communicate with one or more sensors to adjust stimulation and provide closed-loop control of an NST system and/or external respiratory support.

Stimulation of olfactory bulb receptors 118, one or more afferents of the nasal-bronchial reflex, the pre-frontal cortex, suprachiasmatic nucleus, the hypothalamus, cortical respiratory area and/or the brain stem may be accomplished via electrical or magnetic energy transmitted from one or more nasal electrodes 204 and/or one or more dermal electrodes 212. In some embodiments, gas flow from the nasal stimulator (e.g., via one or more outlets 208) may stimulate olfactory bulb receptors 118 or one or more afferents of the nasal-bronchial reflex. FIG. 1B shows a partial cross-sectional view of nasal stimulator 102 inserted into the nasal cavity of a subject 100, along with flow arrows 260 that describe an exemplary flow of gas in a subject.

As can be seen from the flow arrows 260 shown in FIG. 1B, gas may flow from one or more outlets 208 of one or more nasal tubes 206 into the subject 100. The gas may flow through the middle nasal conches 122 or the superior nasal conches 120, and past the olfactory bulb receptors 118.

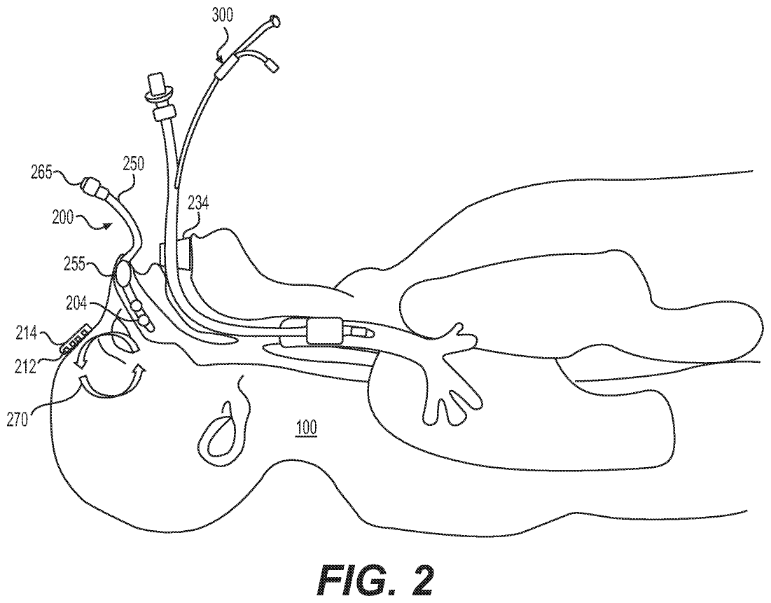

According to one or more embodiments, a nasal stimulator 102 (or any other nasal stimulator of this disclosure) may be used in conjunction with an oral endoscope 300 (see FIG. 2). Oral endoscope 300 may be inserted into the esophagus and/or trachea of a subject 100 to assist the subject 100 in feeding and/or breathing. Various oral endoscopes 300 known in the art may be employed, such as, for example, the esophageal catheters described in U.S. Pat. Pub. 2019/0038894, which is incorporated by reference in its entirety. Other contemplated endoscopes 300 include those used for external respiratory support (e.g., tracheal mechanical ventilation). Endoscope 300 may include a sensor 234 that measures one or more properties of the breath cycle of the patient (e.g., a breathing sensor).

Referring to FIG. 2, a nasal stimulator 200 may include a gas hose 250, one or more occlusion devices 255, one or more nasal electrodes 204, a dermal patch 214, one or more dermal electrodes 212, a sensor 234, and a gas luer 265. An occlusion device 255 (e.g., a balloon) may be configured to be inflated after placement of nasal stimulator 200. For example, a distal end of nasal stimulator 200 may be placed such that one or more nasal electrodes 204 are in the nasal cavity of a subject 100. For example, nasal stimulator 200 may be positioned such that one or more nasal electrodes 204 are proximate to the superior nasal conches 120 and/or the middle nasal conches 122.

After placement of nasal stimulator 200, one or more occlusion devices 255 may be inflated (e.g., with saline, a gas, or another fluid). The occlusion device 255 may expand, upon receipt of fluid, to fill the nasal cavity of the subject 100. For example, when expanded, occlusion device 255 may block one or more passageways of the nasal cavity (e.g., forming an air-tight seal between the nasal cavity and the nasal stimulator 200). Occlusion device 255 may include, for example, a compliant or noncompliant balloon. When deflated, occlusion device 255 may include a deflated or folded balloon. The blocking of one or more passageways with an inflated occlusion device 255 may reduce the gas pressure required to stimulate one or more stimulation targets with gas from the nasal stimulator 200.

As described previously, energy (e.g., electrical or magnetic) may be transmitted from one or more dermal electrodes 212 to one or more other dermal electrodes 212 and/or one or more nasal electrodes 204. Similarly, energy may be transmitted from one or more nasal electrodes 204 to one or more other nasal electrodes 204 and/or one or more dermal electrodes 212. This flow of electrical or magnetic energy, represented by flow arrows 270, may stimulate olfactory bulb receptors, one or more afferents of the nasal-bronchial reflect, the prefrontal cortex, cortical respiratory area and/or the brain stem of subject 100.

Nasal stimulator 200 may include a gas luer 265 at the proximal end of gas hose 250. Gas luer 265 may provide an air-tight connection from the nasal stimulator 200 to a gas source. For example, gas luer 265 may connect gas hose 250 to the gas source and/or the controller. Nasal stimulator 200 may include a regulator (not shown), which may be configured to adjust gas pressure.

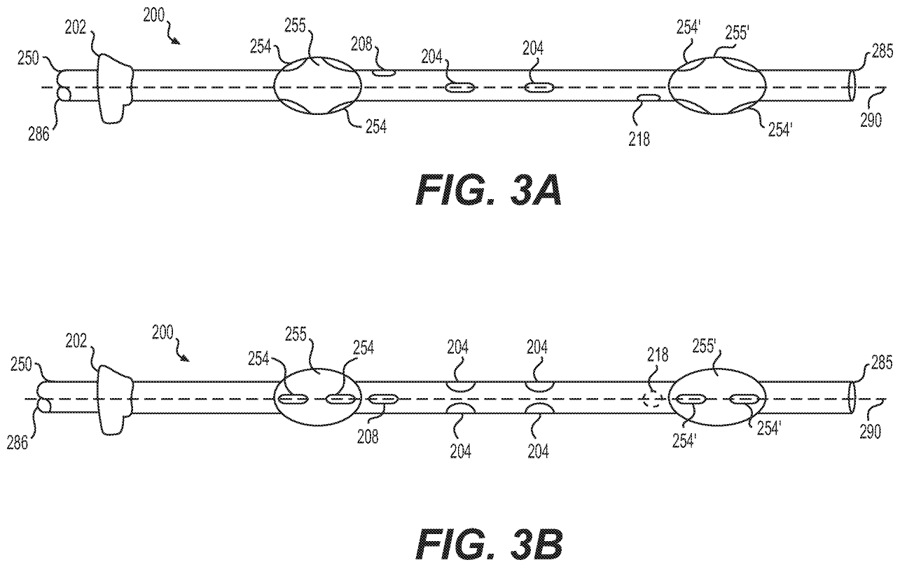

According to one or more embodiments, a nasal stimulator 200 may include any of the features or aspects described herein in relation to one or more other embodiments. One exemplary configuration of a nasal stimulator 200 is shown in FIGS. 3A and 3B. The nasal stimulator 200 shown in FIG. 3B is the same nasal stimulator 200 shown in FIG. 3A, but the entire structure is rotated 90° about a longitudinal axis 290 that extends through the center of a lumen defined by the nasal stimulator 200. Nasal stimulator 200 may include a distal end 285 and a proximal end 286 opposite the distal end 285. The proximal end 286 may be joined to a gas hose 250 via, for example nasal cavity interface 202. The gas hose 250 may extend and connect to a gas source, via, for example, a gas luer 265. The distal end 285 may be closed (e.g., forming a rounded tip; closing one or more lumens defined within nasal stimulator 200) or open (e.g., so that a lumen defined within nasal stimulator 200 is in fluid communication with the nasal passage, through the distal end 285). Nasal stimulators 200 including an open distal end 285 may be configured to deliver gas to a subject via the open distal end 285.

Still referring to FIGS. 3A and 3B, a nasal stimulator 200 may include a nasal cavity interface 202, one or more occlusion devices 255, one or more nasal electrodes 204, and one or more gas outlets 208 and/or gas inlets 218. As described previously, gas outlets 208 may be configured to allow gas to pass from an interior of the nasal stimulator 200 (e.g., a lumen of the nasal stimulator 200) to an exterior of the nasal stimulator 200. Gas inlets 218 may be configured to allow gas to pass from an exterior of the nasal stimulator 200, to an interior of the nasal stimulator 200 (e.g., a lumen of the nasal stimulator 200). All gas outlets 208 and gas inlets 218 may share a single lumen for transport and delivery of gas, or, alternatively, each gas outlet 208 and gas inlet 218 may be connected to a corresponding lumen, where the corresponding lumen not connected to another gas outlet 208 or gas inlet 218. In some embodiments multiple gas outlets 208 or gas inlets 218 may share a lumen for delivery and transport of gas.

Each occlusion device 255 may function similarly to the occlusion devices described in relation to the embodiment shown in FIG. 2. In some embodiments, a nasal stimulator 200 may include two occlusion devices (e.g, occlusion device 255 and 255′). In some embodiments, multiple occlusion devices 255, 255′ may be inflated and/or deflated in combination with each of the other occlusion devices 255, 255′. In addition or alternatively, each occlusion device 255, 255′ may be inflated and/or deflated independently of one or more other occlusion devices 255, 255′. In embodiments, where multiple occlusion devices 255, 255′ are configured to be independently adjusted, nasal stimulator 200 may include multiple lumens for delivery of fluid (e.g., saline, air, etc.) to inflate the occlusion devices 255, 255′.

When occlusion device 255 and occlusion devices 255′ are both inflated, a length of a nasal passage may be closed off (e.g., sealed) between occlusion devices 255, 255′. Gas (e.g., gas for stimulation of an anatomical target) may be passed between gas outlet 208 and gas inlet 218 without leaving the closed off length of the nasal passage, reducing the requisite pressure needed for stimulation of one or more anatomical targets (e.g., olfactory bulb receptors).

Each occlusion device 255, 255′ may further include one or more occlusion device electrodes 254. Placement of electrodes 254, 254′ on an occlusion device 255, 255′ may allow for the electrodes to be closer to tissue (e.g., closer to olfactory bulb receptors 118 or brain stem (not pictured) of a subject 100). Similar to nasal electrodes 204, occlusion device electrodes 254, 254′ may be located at different radial positions about axis 290 of nasal stimulator 200. For example, two or more occlusion device electrodes 254, 254′ of each occlusion device 255, 255′ may be arranged in rows/lines (e.g., lines at different radial positions). For example, an occlusion device 255 may include at least two occlusion device electrodes 254 aligned along a longitudinal axis of the occlusion device 255. Lines of longitudinally aligned occlusion device electrodes 254 may be spaced at different radial positions of occlusion device 255 (e.g., two lines spaced 180° part, three lines spaced 120° apart, or four lines spaced 90° apart). One or more occlusion device electrodes 254 of one occlusion device 255, may be aligned or offset from one or more occlusion device electrodes 254′ of another occlusion device 255′. As described above, energy (e.g., electrical or magnetic) may be passed between two or more electrodes (e.g., nasal electrodes 204, dermal electrodes 212, and/or occlusion device electrodes 254, 254′) to provide stimulation to one or more stimulation targets, such as, for example, olfactory bulb receptors 118, one or more afferents of the nasal-bronchial reflex, the pre-frontal cortex, hypothalamus, cortical respiratory area and/or brain stem.

Placement of one or more gas outlets 208 and/or gas inlets 218 between occlusion devices 255, 255′ may reduce the requisite gas pressure needed to stimulate, for example, olfactory bulb receptors 118. The placement of gas outlet 208 and gas inlet 218 in FIGS. 3A-3B is exemplary, for example, the positions of gas outlet 208 and gas inlet 218 may be interchanged. In some embodiments, gas outlet 208 is radially spaced 180° apart from gas inlet 218, about axis 290. The gas flow from gas outlet 208 to gas inlet 218 may be coordinated with the gas flow from external respiratory support, and/or the subject's innate breath cycle, to enhance to the effectiveness of therapy.

As described above, nasal stimulator 200 may include one or more lumens defined therewithin. For example, one or more lumens may provide for gas flow from the gas source, through nasal stimulator 200 to one or more gas outlets 208 (e.g., distal end 285). Further, the means for inflating one or more occlusion devices 255, 255′ (e.g., saline, air, or another fluid) may be provided from the source (e.g., gas source), through one or more lumens of nasal stimulator 200, to the occlusion device 255, 255′. In some embodiments, the electrical leads for occlusion device electrodes 254, 254′ and/or nasal electrodes 204, may be provided within one or more lumens of nasal stimulator 200. The electrical leads may be passed through one or more lumens containing gas or another fluid, or may be included in one or more separate lumens. The electrical leads may include wires, insulated metal leads, or metal (e.g., printed metal) embedded on and/or within one or more insulative materials.

Embodiments of this disclosure include a stimulation therapy system. For example, the system may be configured to modulate lung compliance, hippocampus activity (e.g., hippocampus neurogenesis, hippocampus astrogenesis, and/or hippocampus inflammation), pre-frontal cortex activity, brain stem activity, trigeminal nerve activity, facial pain, or a combination thereof, of a subject. The system may include an energy source, a nasal interface, and a controller. The energy source may be a source of electrical energy, a source of mechanical energy (e.g., a pressurized gas source), or another source of stimulation energy. Further, the energy source may be external of the subject.

In some embodiments, a stimulation therapy system may include one or more occlusion devices and/or a securement means. The one or more occlusion devices may be actuatable (e.g., inflatable, expandable). One or more occlusion devices may be configured to prevent, or otherwise minimize, stimulation from the system (e.g., gas flow) from entering the lungs of the subject. The securement means may function either on the outside of the subject (e.g., straps wrapped around the subject's head) or inside the subject. The securement means may allow for one or more electrodes to be affixed in contact with the inner nose and/or the forehead of a subject. A system may further include a dermal patch that is configured to be affixed to a subject's skin. The dermal patch may include one or more electrodes (e.g., an electrode array), such as, for example, electrodes configured to deliver electrical or magnetic stimulation.

A stimulation therapy system may include, be in communication with, or be configured to communicate with, one or more external respiratory support devices. Exemplary external respiratory support devices include, but are not limited to, mechanical ventilators, CPAP machines, high-low oxygen masks, phrenic nerve stimulation devices, and/or an oral endoscope (e.g., a tracheal cannula or an endotracheal tube).

Stimulation energy may be directed through the nasal interface. For example, gas may be directed from a gas source, through the nasal interface (e.g., through an outlet connected to a nasal interface), to a stimulation target. In addition or alternatively, electrical energy may be directed from a source of electrical energy, through the nasal interface (e.g., through a lead in the nasal interface that is electrically coupled to an electrode), to a stimulation target. Stimulation targets may include, but are not limited to, olfactory bulb receptors, a vagus nerve, a trigeminal nerve, a pre-frontal cortex, a suprachiasmatic nucleus, a hypothalamus, cortical respiratory area, a brain stem, one or more connected nerves and their afferents and efferents, or a combination thereof. Stimulation energy may pass through, and/or adjacent to, one or more tissues of the nasal cavity.

The controller may manage the delivery of stimulation, such as, for example, mechanical stimulation via gas flow, electrical stimulation, magnetic stimulation, mechanical stimulation via intranasal cavity pressure modulation, thermal stimulation, infrared stimulation, electromagnetic stimulation, or a combination thereof. Stimulation may be delivered, as coordinated by the controller, from multiple sources, such as, for example, electrical stimulation from multiple electrodes, gas flow from multiple gas flow sources, infrared stimulation from multiple infrared input and output sources, and/or magnetic stimulation from a multi-dimensional magnetic field. The controller may coordinate information between one or more sensors, energy sources, other components of the system, and one or more external respiratory support devices. The controller may also interface with one or more external respiratory support devices, such as, for example, a mechanical ventilator, to control delivery of external respiratory support. The one or more sensors may measure energy delivery (e.g., stimulation energy delivered), an electrical activity and/or potential representative of nerve or muscle activity, a distance between two sources of infrared energy, a flowrate, one or more diameters of one or more occlusion devices, an absorbance, a transmittance, a reflectance, an impedance, a magnetic field direction, a magnetic field magnitude, a pressure, or a combination thereof. The controller may be configured to deliver stimulation energy in synchronization with a breath cycle, such as, for example, a breath cycle of an external respiratory support device and/or a subject's innate breath cycle.

The one or more sensors may communicate one or more measured properties to the controller. The controller may include, or may be in communication with, a processor that analyzes signals from the one or more sensors. The controller may use information from one or more sensors to adjust stimulation parameters. Stated another way, the stimulation therapy system may function as a component of a closed-loop system. Alternatively, or in addition, a stimulation therapy system may include a monitor or other display device, and a user may receive data from the stimulation therapy system and may adjust stimulation parameters based on the received data.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and example be considered as exemplary only.

1. A stimulation device comprising:
a tube defining a lumen therein and a gas outlet, wherein the gas outlet is configured to allow gas to pass from the lumen to an exterior of the tube;
one or more first electrodes associated with the tube;
one or more second electrodes; and
a nasal cavity interface proximal of the gas outlet and the one or more first electrodes.

2. The stimulation device of claim 1, further comprising a gas hose connected to the nasal cavity interface, wherein the gas hose includes:
a distal end that allows gas to flow from the gas hose to the gas outlet; and
a proximal end configured to receive gas into the gas hose from a gas source.

3. The stimulation device of claim 2, further comprising one or more metal leads connected to the one or more first electrodes, wherein the one or more metal leads are disposed within the nasal cavity interface and within the gas hose.

4. The stimulation device of claim 1, further comprising an inlet port configured to allow gas to pass from the exterior of the stimulation device to the interior of the stimulation device.

5. The stimulation device of claim 1, wherein the gas outlet is a first gas outlet, and the stimulation device further comprises a second gas outlet configured to allow gas to pass from the lumen to the exterior of the stimulation device.

6. The stimulation device of claim 5, further comprising an occlusion device configured to expand upon receipt of a fluid transmitted through the nasal cavity interface, wherein the occlusion device is between the first gas outlet and the second gas outlet.

7. The stimulation device of claim 6, wherein the occlusion device is a first occlusion device, and the stimulation device further comprises a second occlusion device configured to expand upon receipt of a fluid transmitted through the nasal cavity interface.

8. The stimulation device of claim 7, wherein the first occlusion device supports the one or more first electrodes, and the second occlusion device supports the one or more second electrodes.

9. The stimulation device of claim 5, wherein the tube is a first nasal tube, and the stimulation device further comprises a second nasal tube, wherein:
the one or more first electrodes are disposed on a surface of the first nasal tube;
the one or more second electrodes are disposed on a surface of the second nasal tube;
the first gas outlet is disposed on a distal end of the first nasal tube; and
the second gas outlet is disposed on a distal end of the second nasal tube.

10. The stimulation device of claim 9, wherein the one or more first electrodes include a first line of electrodes perpendicular to the first gas outlet, and a second line of electrodes parallel to the first line of electrodes; and
the one or more second electrodes include a third line of electrodes perpendicular to the second gas outlet, and a fourth line of electrodes parallel to the third line of electrode.

11. The stimulation device of claim 1, further comprising:
a sensor; and
a controller in communication with the sensor, the one or more first electrodes, and the one or more second electrodes, wherein the controller is configured to adjust an amount of gas passing through the gas outlet.

12. The stimulation device of claim 11, wherein the controller is further configured to:
analyze data collected by the sensor to determine a breath cycle; and
adjust an amount of gas passing through the gas outlet based on the determined breath cycle.

13. The stimulation device of claim 11, wherein the controller is configured to deliver signals to the one or more first electrodes and the one or more second electrodes, such that an electrical field is generated between the one or more first electrodes and the one or more second electrodes.

14. The stimulation device of claim 1, further comprising a dermal patch supporting the one or more second electrodes and configured for placement on skin.

15. The stimulation device of claim 11, wherein the sensor is supported on an endoscope.

16. A stimulation device comprising:
a tube defining a lumen therein and a gas outlet, wherein the gas outlet is configured to allow gas to pass from the lumen to an exterior of the tube;
one or more first electrodes associated with the tube;
a nasal cavity interface proximal of the gas outlet and the one or more first electrodes;
a sensor; and
a controller in communication with the sensor, wherein the controller is configured to adjust an amount of gas passing through the gas outlet.

17. The stimulation device of claim 16, further comprising a dermal patch supporting one or more second electrodes and configured for placement on skin.

18. The stimulation device of claim 17, wherein the controller is configured to deliver signals to the one or more first electrodes and the one or more second electrodes, such that an electrical field is generated between the one or more first electrodes and the one or more second electrodes.

19. The stimulation device of claim 16, further comprising an occlusion device configured to expand upon receipt of a fluid transmitted through the nasal cavity interface, wherein the occlusion device is between the gas outlet and the nasal cavity interface, and the occlusion device supports the one or more first electrodes.

20. The stimulation device of claim 16, further comprising an inlet port configured to allow gas to pass from an exterior of the stimulation device to an interior of the stimulation device; and
a gas hose connected to the nasal cavity interface, wherein the gas hose includes:
a distal end that allows gas to flow from the gas hose to the gas outlet; and
a proximal end configured to receive gas into the gas hose from a gas source.

21. The stimulation device of claim 20, wherein the gas source includes a mixture of oxygen and one or more other gases.
